(19) United States
(12) Patent Application Publication
(10) Pub. No.: US 2021/0393911 A1
(43) Pub. Date: Dec. 23, 2021

(54) SMART ENDOTRACHEAL TUBE

(71) Applicant: [AI]rway, Inc., Oakland, CA (US)

(72) Inventors: Isaac Cohen, Oakland, CA (US); Ori Cohen, Albertson, NY (US); Carlos Milla, Redwood City, CA (US); Yossi Chay, Berkeley, CA (US)

(73) Assignee: [AI]rway, Inc., Oakland, CA (US)

(21) Appl. No.: 17/230,756

(22) Filed: Apr. 14, 2021

Related U.S. Application Data
(60) Provisional application No. 63/042,729, filed on Jun. 23, 2020.

(51) Int. Cl. A61M 16/04; A61B 1/05; A61B 1/267; A61M 16/00

(52) U.S. Cl. CPC A61M 16/0488; A61B 1/05; A61B 1/267; A61M 2205/3331; A61M 16/0051; A61M 2205/50; A61M 16/0434

(57) ABSTRACT

A system and method for ventilating the lungs of a patient. The system includes a smart tube for intubation comprising a tip and a cuff. The system also includes one or more cameras, coupled with the smart tube, for providing relative positional intubation measurements of the patient and/or providing intubation images of the patient. Further, the system includes one or more sensors, coupled with the smart tube, for sensing the inflation of the cuff and/or sensing pressure of the cuff. The system for ventilating additionally includes a computing system, coupled with the camera, the at least one sensor and the smart tube, for computing programmed feedback based on the relative positional intubation measurements, and/or the intubation images, and/or the inflation of the cuff, and/or the pressure of the cuff.

### BACKGROUND

### Field of the Disclosure

The present disclosure generally relates to medical equipment generally, and more particularly, to a smart endotracheal tube.

### Description of Related Art

This section intends to provide a background discussion for a clear understanding of the disclosure herein but makes no claim nor any implication as to what may the relevant art for this disclosure.

Various medical equipment may be currently employed in supporting impaired human breathing, most commonly referred to ventilators. A ventilator may be defined as a device machine that provides mechanical ventilation by moving breathable air into and out of the lungs, thereby delivering breaths of an oxygen mix to a patient impaired or physically unable to breathe or breath sufficiently. While numerous ventilator designs exist, the most advanced systems rely on computer controlled designs though hand-operated bag valve mask construct may still in use.

With the recent rise of Coronavirus, also known as Covid 19, the shortage of ventilators has become more glaringly more apparent to public health and safety experts as well as the general public. The most severely impaired Covid 19 patients require mechanical ventilation once admitted into intensive care units in hospitals. Naturally, ventilators may also be found, for example, in mobile emergency medicine, in-home care, as well as in conjunction with anesthesia machines.

Ventilators may be currently implemented using an electro-mechanical system to push air through the trachea and into a patient's lungs. These systems rely on motors or pumps to effectively allow a patient to breath mechanically. Electro-mechanical systems like motors and pumps have a predictable fail rate time, require maintenance, consume high amounts of energy, generate heat waste and add bulk to a ventilator design. Consequently, many ventilation machines use electric motors and brushless driven turbine to control the pressurized air flow during both inhalation and exhalation of the lungs, without depending on pressurized gas supply.

Mechanical ventilation also requires the use of endotracheal tubes (ETT). Referring to FIG. 1, a known ETT 10 may illustrated. ETTs may disposable devices, and as illustrated, manually inserted through a patient's mouth, passing the epiglottis, glottis, vocal cords and into the trachea at a short distance proximal to the main carina, otherwise known as tracheal carina. Surrounding the distal end of ETT 10 may an inflatable cuff 20 intended to secure the smart tube's placement and position in the patient's trachea. Inflatable cuff 20 may also intended to prevent leaks in any gap between the smart tube and the trachea.

The necessity for intubating the patient with an ETT to establish mechanical ventilation may be driven by a number of medical circumstances. One such an example may respiratory failure in patients undergoing surgeries using general anesthesia. As recent as 2014, there may data estimating about 15 million, or 30%, of all surgeries in the US required an ETT with medical patients. Ventilation and ETT intubations may also routinely used in respiratory failure cases caused by acute lung injury or parenchymal lung disease. In emergency care, upwards of 30% of intubations using an ETT and ventilator may be attributed to trauma.

Currently, ETT insertions may conducted by medical professionals using a laryngoscope. Laryngoscopes including one of various blade designs to enable the lifting of the epiglottis to allow the ETT access and afford visualization of the epiglottis, glottis and vocal cords. Common designs used in laryngoscopes may include the Macintosh and the Miller blades. Besides potential damage to the epiglottis, the laryngoscopic blades may also injure the maxillary incisors or gingiva if used as a lever in ETT insertion.

While most surgical and intubation risks may statistically low, there may some acute issues that may potentially arise if a patient remain on mechanical ventilation for a prolonged time period. These risks, for exemplary purposes, may include: trauma to the teeth, mouth, tongue or larynx; accidental intubation in the esophagus instead of the trachea; inadvertent deep intubation into a mainstem bronchus; trauma to the trachea; excessive bleeding; inability to be weaned from the ventilator thereby requiring a tracheostomy; aspirating (e.g., inhaling) vomit, saliva or other fluids while intubated; pneumonia, if aspiration occurs; sore throat; hoarseness; and erosion of tracheal tissue (with prolonged intubation). Of the above noted risks, tube misplacements are, unfortunately, common. In one 1994 study, following 12 months of training, it was note that 46% of intubations were misplaced and required repositioning.

ICU patients may require urgent ventilation by an anesthesiology trained expert. Even this with 6 months training, there may an 8% intubation insertion rate performed by an expert that may be deemed “difficult”—currently defined as requiring two or more attempts at ETT placement.

Additionally, a 3% morbidity rate within 30 minutes of intubation has been noted as recently as 2014. According to this same source, having a faculty anesthesiologist present during ICU intubations has shown marked lower rate of complication rate—21.7% versus 6.1%. Problems with operation room ICU intubations have been shown in 2014 to occur in 11.3% of incidences. Of these problematic 2014 cases, 38% of patients suffered serious complications, including profound hypoxia, cardiac collapse, cardiac arrest, and death.

The duration of mechanical ventilation has been viewed as an indicator of significant health complications. This may also true in terms of health-care cost. The American Society of Anesthesiologists Closed Claims database has shown as recent as 2011 that for the decade from 2000 to 2010, 17% of all surgery-related claims were as a result of airway mis-management.

Prolonged use of ETT with inflated cuff may one of the major multifactorial causes of complications with mechanical ventilation. ETT cuff pressure may critical to proper and safe functioning of ventilation. Low ETT cuff pressure results in leakage and aspiration of GI fluids and flora. High ETT cuff pressure may result in trauma to the trachea. ETT cuff pressure estimated by palpation may be much higher than measured or what medically optimal—e.g., a mean ETT cuff pressure measured after estimation by palpation of the pilot balloon 58.4±31.7 cm H₂O before adjustment, wherein the highest measurement may be 285.5 cm H₂O, and 27.1±4.2 cm H₂O after adjustment. Traditionally, clinicians have measured intra-cuff pressure as a surrogate for contact pressure. Intra-cuff pressures greater than 48.9 cm H₂O may impede capillary blood flow and potentially damage the mucosal lining of the trachea from long term stenosis. It also has been considered traditional that intra-cuff pressures of less than 25.5 cm H₂O may increase the risk of aspiration of mucosal secretions, causing a higher incidence of ventilator associated pneumonia.

Despite the above risks, micro-aspiration of secretions into the lower respiratory tract contaminated by bacteria have been associated with the pathogenic mechanism for ventilator associated pneumonia (“VAP”). Despite known ETT designs, cuff pressure at placement and changes during ventilation, VAP remains a common post-operative complication, mainly due to inappropriate seal between the ETT cuff and the patient's trachea. Inappropriate seal typically occurs from over and under inflation and may result in ETT movement during ventilation and dislocations, thereby causing accidental extubation and self-extubation.

Consequently, it may be known that ETT intubation may results in numerous potential complications including patient morbidity. Current ETT designs do not allow for accurate and safe passage and placement in the trachea. Position and cuff pressure during ventilation may increase the risks associated with ventilation. These issues and others may be exaggerated during prolonged ventilation.

### SUMMARY

The present disclosure includes a system and method for ventilating the lungs of a patient using smart endotracheal tube.

In one aspect, a system for ventilation the lungs of a patient may disclosed. The system comprises an a smart ETT, hereinafter smart tube, for intubation having a tip and a cuff, one or more cameras, coupled with the smart tube, one or more sensors, coupled with the smart tube, and a computing system, coupled with the one or more cameras, the one or more sensors and the smart tube. The one or more cameras provides relative positional intubation measurements of the patient and/or intubation images of the patient. The one or more sensors sense the inflation of the cuff and/or pressure of the cuff. Moreover, the computing system computes programmed feedback based on the relative positional intubation measurements, and/or the intubation images, and/or the inflation of the cuff, and/or the pressure of the cuff.

In yet another aspect of the disclosure, the one or more sensors measure the patient's tracheal wall perfusion to optimize cuff pressure control.

In another aspect of the disclosure, the programmed feedback generates computational real time positional guidance in the intubation of the smart tube.

In yet another aspect of the disclosure, the computational real time positional guidance generates at least one of misplacement signaling and proper placement signaling.

In still another aspect of the disclosure, the computing system generates a displacement alert signal if the computing system measures the computational real time positional guidance outside a programmed location range.

In yet another aspect of the disclosure, the computing system generates the proper placement signaling if the computing system measures the computational real time positional guidance within a programmed location range of the tip of the smart tube relative to the patient's carina.

In yet another aspect of the disclosure, the computing system further comprises a programmed servo system for positioning the smart tube relative to the patient's carina within the programmed location range in response to the proper placement signaling, and/or repositioning the smart tube relative to the patient's carina within a programmed repositioning location range, in response to the displacement alert signaling.

In yet another aspect of the disclosure, the programmed feedback generates computational real time cuff pressure signaling.

In yet another aspect of the disclosure, the computing system generates a real time cuff pressure signaling alert if the computing system measures the computational real time cuff pressure signaling outside a programmed cuff pressure range.

In yet another aspect of the disclosure, the computing system further comprises a programmed servo system for maintaining the cuff pressure within the programmed cuff pressure range in response to computational real time cuff inflation signaling.

In yet another aspect of the disclosure, the smart tube, the one or more cameras, the one or more sensors, and the computing system may be integrated within a singular housing.

In another one aspect, a method for ventilation the lungs of a patient may disclosed. The method comprises the step of intubating the patient with a smart tube having a tip and a cuff. Further, the method comprises providing relative positional intubation measurements of the smart tube relative to the patient. The method also comprises the step of sensing pressure of the cuff. Moreover, the method comprises the step of computing programmed feedback based on the relative positional intubation measurements and/or the pressure of the cuff.

In another aspect of the disclosure, the step of computing programmed feedback comprises the step of generating computational real time positional guidance in the intubating of the smart tube.

In yet another aspect of the disclosure, the step of generating computational real time positional guidance in the intubating of the smart tube comprises the step of generating misplacement signaling and/or proper placement signaling.

In still another aspect of the disclosure, the method further comprises the steps of measuring the computational real time positional guidance with a programmed location range, and generating a displacement alert signal if the computational real time positional guidance falls outside the programmed location range.

In yet another aspect of the disclosure, the step of measuring the computational real time positional guidance within the programmed location range comprises the step of measuring the tip of the smart tube relative to the patient's carina.

In yet another aspect of the disclosure, the method further comprises the step of positioning the smart tube relative to the patient's carina within the programmed location range in response to the proper placement signaling, and/or repositioning the smart tube relative to the patient's carina within a programmed repositioning location range, in response to the displacement alert signaling.

In yet another aspect of the disclosure, the step of computing programmed feedback comprises the step of generating computational real time cuff pressure signaling.

In yet another aspect of the disclosure, the method further comprises the step of measuring the computational real time cuff pressure signaling with a programmed pressure range, and/or generating a real time cuff pressure signaling alert if the computational real time cuff pressure signaling falls outside programmed pressure range.

In yet another aspect of the disclosure, the method further comprises the step of maintaining the cuff pressure within the programmed cuff pressure range in response to computational real time cuff inflation signaling.

In yet another aspect of the disclosure, the method further comprises the step of acquiring tracheal wall perfusion information at the cuff.

In yet another aspect of the disclosure, the method further comprises a step of real time computing of a patient's tracheal wall perfusion information relative to cuff pressure.

### BRIEF DESCRIPTION OF THE DRAWINGS

The present disclosure and its various features and advantages can be understood by referring to the accompanying drawings by those skilled in the art relevant to this disclosure. Reference numerals and/or symbols may be used in the drawings. The use of the same reference in different drawings indicates similar or identical components, devices or systems. Various other aspects of this disclosure, its benefits and advantages may be better understood from the present disclosure herein and the accompanying drawings described as follows:

FIG. 1 illustrates a known ETT for intubation into a patient;

FIGS. 2(a)-(e) illustrates an aspect of the present disclosure; and

FIGS. 3-11 illustrate additional aspects of the present disclosure.

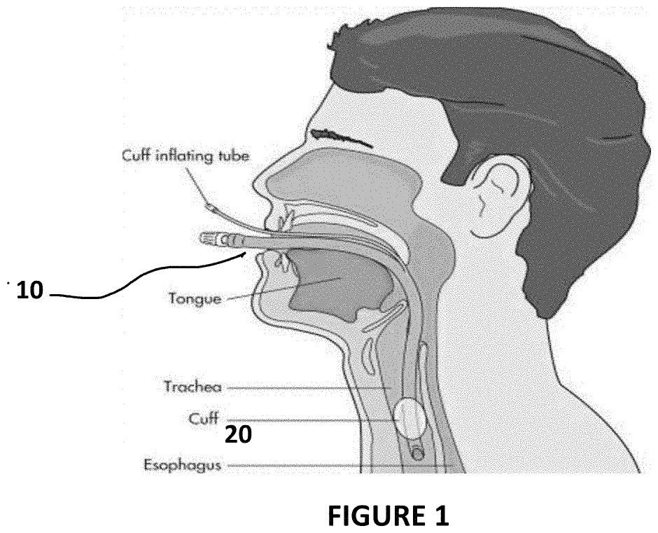

### DETAILED DESCRIPTION

The present disclosure includes a system and method for ventilating the lungs of a patient using smart endotracheal tube.

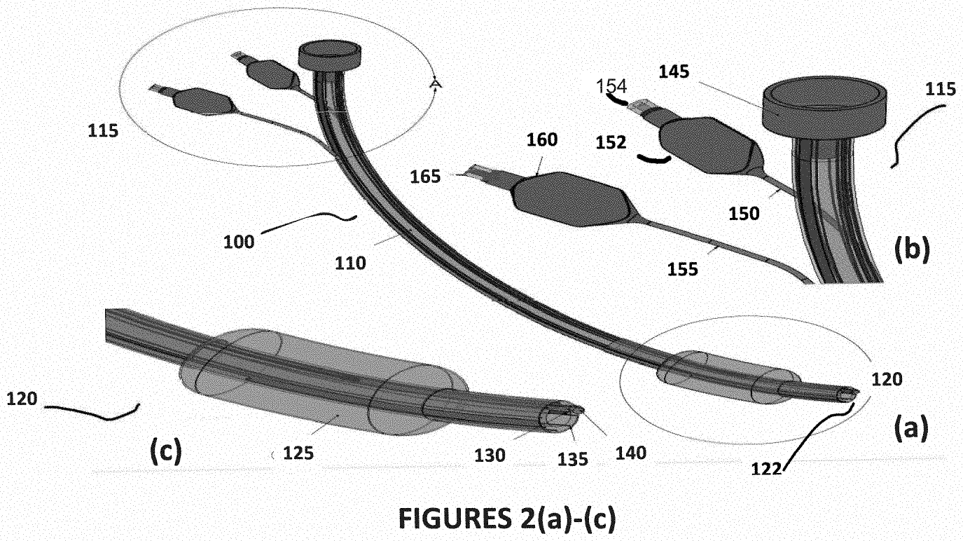

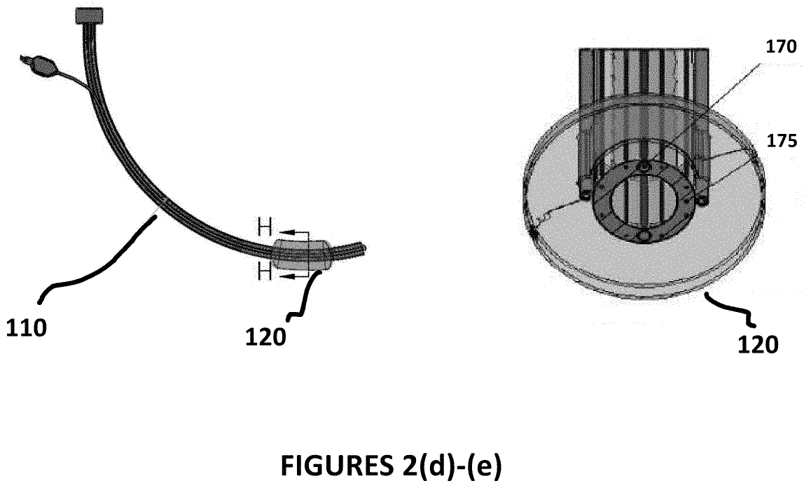

Referring to FIG. 2(a)-(e), a first aspect of the present disclosure may be illustrated. FIG. 2(a) shows a system for ventilation the lungs of a patient using a smart endotracheal tube 100, hereinafter referred as smart tube. Smart tube 100 includes an endotracheal tube (“ETT”) having many aspects that overcome the known challenges of existing solutions.

Smart tube 100 comprises a flexible duct or pipe 110 to achieve the purposes of an intubation in ventilation. It may be apparent to skilled artisans that smart tube 100 may have other applications beyond intubation as well as in support of ventilation of a patient's lungs including endoscopic, colonoscopic and laparoscopic uses. Flexible pipe 110 may be realized by various material including PVC.

Coupled with flexible pipe 110, smart tube 100 includes an input assembly 115. Input assembly 115 may be shown in greater detail in FIG. 2(b). For the purposes of the present disclosure, intubation assembly 120 including a tip 122 at the end of smart tube 100 to achieve the ambition of “smart” intubation, as may be more clearly disclosed.

Smart tube 100 also includes an intubation assembly 120 on the distal end opposite that of input assembly 115. Intubation assembly 120 may be shown in greater detail in FIG. 2(c). For the purposes of the disclosure, intubation assembly 120 may the end of smart tube 100 that may be inserted into the patient's trachea and include various novel and inventive elements to achieve the ambition of smart intubation, as may be more clearly disclosed.

Referring to FIG. 2(b), input assembly 115 may illustrated in an expanded view to shows its aspects in greater detail. As depicted here, input assembly 115 includes a connector 145 positioned at the further end of assembly 115. Connector 145 may be included for coupling with various medical equipment, such as a mechanical ventilator (not shown).

Input assembly 115 further includes a camera balloon cover tube 150. Camera balloon cover tube 150 functions to pass air to flexible duct 110 and thereby enable the proper insertion of smart tube 100 into the patient during an intubation, for example. Camera balloon cover tube 150 may be integrated into and with smart tube 100 and flexible pipe 110 at any point in smart tube 100. In one aspect of the invention, camera balloon cover tube 150 may integrated with flexible pipe 110 before flexible pipe 110 reaches the back of the patient's tongue.

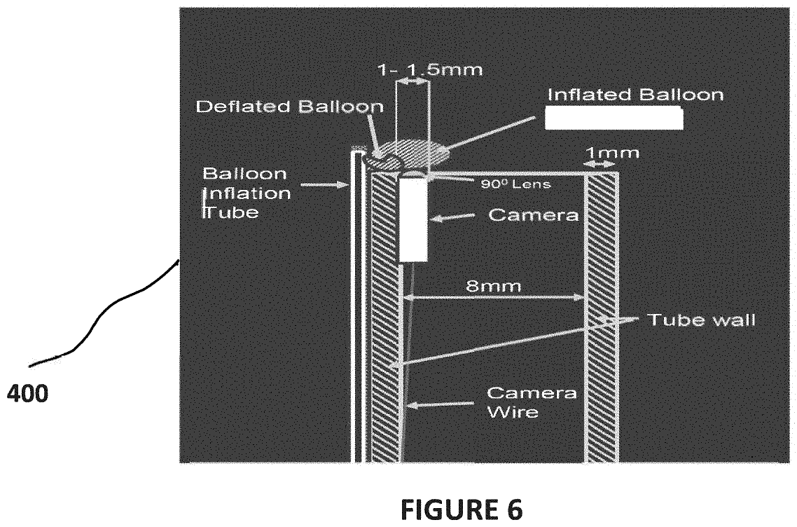

Further, camera balloon cover tube 150, as part of input assembly 115, includes a pilot balloon 152. Pilot balloon 152 stores and passes air through camera balloon inflation tube 150. Pilot Balloon 152, in conjunction camera balloon cover tube 150, enables the insertion of smart tube 100 during intubation, for example. Pilot balloon 152 may inflate camera cover balloon 140 as shown in FIG. 2(c), and more readily, depicted in as cover 400 in FIG. 6. Pilot balloon 152 allows fingertip pressure sensing in camera cover balloon 140 and again, 400, as illustrated in FIG. 6.

To prevent too much air from passing into smart tube 100 from balloon 152 via camera inflation cover tube 150, input assembly 115 also includes an inflation port and pressure release monitor valve 154. Inflation port and pressure release monitor valve 154 manages the air pressure sensing possibility flowing into camera cover balloon 140—or 400 as illustrated in FIG. 6—and through the smart tube 100 and into camera cover balloon into 140 or 400 in FIG. 6 during intubation. In one aspect of the invention, port and pressure release valve 154 includes a spring valve system, for example, though other solutions may become apparent to skilled artisans upon understand the present disclosure.

Additionally, input assembly 115 also includes a cuff inflation tube 155. Cuff inflation tube 155 functions to pass air to and through flexible duct 110 to expand intubation assembly 120 generally and, more particularly, an inflatable cuff 125, shown in greater detail in FIG. 2(c). Cuff inflation tube 155, in concert with other elements detailed below, secures the placement and position of inflatable cuff 125 relative to the patient's trachea. As may be understood from the disclosure below, the air flowing via cuff inflation tube 155 enables inflatable cuff 125 to inflate to desirable size, thereby preventing leaks in any gap between smart tube 100 and the patient's trachea. Cuff inflation tube 155 may be integrated into and with smart tube 100 and flexible pipe 110. In one aspect of the invention, camera balloon cover tube 150 with flexible pipe 110 may integrated at the distal opening of smart tube 100.

Cuff inflation tube 155, as part of input assembly 115, includes a cuff balloon 160. Cuff balloon 160 stores and passes air through cuff inflation tube 155. Cuff pilot balloon 160, in conjunction cuff inflation tube 155, enables inflatable cuff 125 to inflate to desirable size, thereby preventing any leaks cause by a gap(s) between smart tube 100 and the patient's trachea.

To prevent too much air from passing into smart tube 100 from cuff pilot balloon 160 via cuff inflation tube 155, input assembly 115 also includes a cuff monitor inflation port and pressure release valve 165. Cuff inflation port and pressure release monitor valve 165 manages the reservoir of air for tactile sensing of an inflatable cuff 125, best shown in FIG. 2(c), flowing into cuff balloon 160. In one aspect of the invention, value 165 includes a spring value system, for example, though other solutions may become apparent to skilled artisans upon understand the present disclosure.

Referring to FIG. 2(c), intubation assembly 120 may illustrated in an expanded view to shows its aspects in greater detail. As depicted here, input assembly 120 includes inflatable cuff 125. As noted hereinabove, inflatable cuff 125 may be intended to be inflated, by the medical professional, to desirable size to prevent leaks from any gap between smart tube 100 and the patient's trachea. This may insure proper and continued intubation during ventilation as detailed herein.

As shown, intubation assembly 120 includes a number of additional elements to realize smart tube 100. Smart tube 100 includes and may be coupled with one or more cameras 135. Camera 135, as part of smart tube 100, provide images of intubation assembly 120 back up through flexible pipe 110 and assembly 115 to the medical professional. This enables the professional to inspect the positioning of smart tube 100 during insertion and intubation as well as monitor its relative positioning should smart tube 100. As smart tube 100 may move from their final intubation position during ventilation for various reasons, camera 135 may can provide immediate location information to the medical professional and prevent injury or harm to the patient. In one aspect of the disclosure, camera 135 provides a relative positional intubation measurement data of the intubation assembly 120 in the patient.

Intubation assembly 120 moreover includes at least one sensor and light source 130. Sensor and light source 130 coupled with smart tube 100. In one aspect of the disclosure, sensor and light source 130 provide light, in through flexible pipe 110 to enable camera 135 to generate images. In another aspect of the disclosure, sensor and light source 130 monitor the positioning of smart tube 100 relative towards it desired location for the medical professional. Sensor and light source 130, in another aspect of the disclosure, may detect the misplacement of smart tube 100.

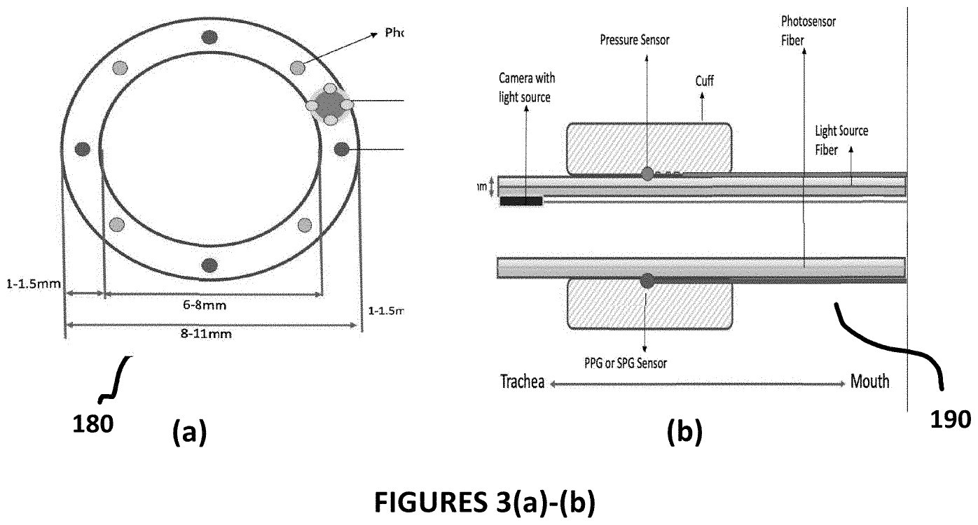

In one aspect of the disclosure, pressure and perfusion sensor 190, best illustrated in FIG. 3(b), include least one pressure sensor and at least one perfusion sensor. The pressure sensor in conjunction with the perfusion sensor allows for measuring and monitoring the inflation and/or pressure monitoring of inflatable cuff 125.

By using the perfusion sensor, the pressure of inflatable cuff 125 may be controlled in response to tracheal perfusion. In yet another aspect of the disclosure, the at least one sensor 190 may include a photoplethysmography (“PPG”) sensor to measure tracheal wall perfusion.

In a further aspect, a control unit with a display (not shown) may provide real time information to the medical professional as well as, in the alternative or in conjunction, enable the remote acquisition of this real time information.

Intubation assembly 120 also includes a camera cover balloon 140. Camera cover balloon 140 assists in the final positioning of camera 135 and light source and sensor 130. It one approach, camera cover balloon 140 has access to the air flow from port 154 to pilot cuff balloon 152 through camera balloon cover tube 150 for inflation. Camera cover balloon 140 protects camera 135 from fogging and mucus debris while positioned in the patient's trachea. Alternatively, camera cover balloon 140 may be realized by spraying saline or similar solution that may spray from the reservoir through camera balloon cover tube 150 to protect camera 135 from fogging and mucus debris via the patient. In this alternative, camera cover balloon 140 may also provide drug delivery directly to the patient.

Referring to FIGS. 2(d) and 2(e), another aspect of the disclosure may be illustrated. In FIG. 2(d), smart tube 100 may depicted as hereinabove with intubation assembly 120. Here, the “H” or horizontal perspective arrows depicted within intubation assembly 120 in FIG. 2(d) may reflected in FIG. 2(e).

Referring to FIG. 2(e) show a perspective cross sectional view of intubation assembly 120. Intubation assembly 120, as shown, includes a camera cable 175 for communicating images and/or real time positional data of smart tube relative to the patient's trachea from camera 135, as illustrated in FIG. 2(c), to the medical professional at the opposite distant end of intubation assembly 120 of smart tube 100 outside the patient.

Moreover, intubation assembly 120 includes a sensor 170. In one aspect of the disclosure, sensor 170 may include on or more of the following: a photoplethysmography (PPG) sensor; a specific gravity (SPG) sensor; and a pressure sensor 190 as best shown in FIG. 3(b). As noted herein, sensor 170 may sense the inflationary state of inflatable cuff 125, measuring and monitoring the inflation and/or pressure of inflatable cuff 125, as well as monitoring the positioning of smart tube 100 relative towards it desired location for the medical professional, while detecting the misplacement of smart tube 100.

In another aspect of the disclosure, which may be further apparent the hereinbelow, smart tube 100 also includes a computing system. The computer system may be integrated with smart tube 100, camera 135, light and sensor 130 in a single housing.

In one aspect, the aforementioned computer system may be positioned at the opposite distant end of intubation assembly 120 of smart tube 100 outside the patient. The computer system may be coupled with camera 135, light source and sensor 130 as well as smart tube 100. The computer system provides computational assistance and programmed feedback based on inputs from camera 135, light source and sensor 130. In one aspect of the disclosure, the computer system generates feedback in response to any of the following data sources: the relative positional intubation measurements of smart tube 100; and/or intubation images of the smart tube 100 as identified through image processing techniques; and/or the inflation of inflatable cuff 125; and/or the pressure of the inflatable cuff 125.

It should be noted the programmed feedback generated by the computer system may, in on aspect of the disclosure, include computational real time positional guidance in the intubation of smart tube 100 to the medical professional. This computational real time positional guidance may generate, in one aspect, misplacement signaling and/or proper placement signaling to the medical professional. In another aspect, a displacement alert signal may be generated by the computing system if the computational real time positional guidance measured by camera 135 and light source and sensor 130 fall outside a desired range. In this instance, a medical professional may program the desired range based on the patient's physiology amongst other potential variables. Furthermore, the computing system may, in yet another aspect of the disclosure, generate the proper placement signaling if the computational real time positional guidance within a programmed location range of tip 122 of smart tube 100 relative to the patient's trachea and carina.

In another aspect of the disclosure, the programmed feedback generated by the computer system may be computational real time cuff pressure signaling. This real time cuff pressure signaling, though the computer system, may generate an alert if the computational real time cuff pressure signaling measure by the computer system falls outside a programmed cuff pressure range. As with location, a medical professional may program the desired range based on the patient's physiology amongst other potential variables.

In another aspect of the disclosure, the computing system performs additional functions to provide assistance to the medical professional. In one example, the computing system also a programmable servo system or some similar implementation apparent to skilled artisans upon reviewing the disclosure herein, to achieve computer controlled maintenance of smart tube 100, its location and positioning, as well as aspects of inflatable cuff 125. Here, the computing system enables the proper position of the smart tube relative to the patient's trachea and carina within the programmed location range in response to the proper placement signaling. Similarly, the computing system enables the repositioning of smart tube 100 relative to the patient's trachea and carina within the programmed repositioning location range, in response to the displacement alert signaling. Likewise, the programmed servo system may adjust the cuff pressure of inflatable cuff 125 to fall within the programmed cuff pressure range in response to computational real time cuff inflation signaling. The programmable servo system may use machine learning and adaptive statistical algorithms to realize these features. Further, the aforementioned programmable servo system may be realized by a robotic surgical tool mechanism for insertion of smart tube 100. The system is monitoring two main parameters through the intubation process: tube position and cuff pressure. Monitoring is done using one or more cameras, cuff pressure sensor and PPG. The captured images and sensor readouts are fed to one or more machine learning engines with pre-trained models. To enhance the accuracy of the models, some other parameters could be used as inputs to these models, such as: age, gender, pre-existing conditions, blood pressure, SpO2, heart rate and more. The output of the machine learning engines will indicate: correct positioning of the tube and optimal cuff pressure. During the intubation process, the doctor monitors in real time the tube position and once located, the cuff pressure. All the data that is collected (images and other sensor readouts) is annotated naturally since for each set of readouts, the operator decides if the tube position and cuff pressure are correct or needs to be fixed. Those decisions by the operator are the same as the autonomous machine learning models have to make periodically throughout the intubation period. All these annotated datasets, collected from many units can be uploaded to the cloud and can be used to retrain the models and increase their precision and accuracy. Each retraining session yields a set of coefficients that can be shared with subscribed units and update their machine learning models.

Referring to FIGS. 3(a)-3(b), another aspect of the present disclosure may be illustrated. In FIG. 3(a), another cross-section of inflatable cuff 180 may be shown. On the peripherical ring of inflatable cuff 180, photosensors may depicted in green, light sources may depicted in red and a camera may be depicted grey encapsulated by yellow.

Referring to FIG. 3(b), a cross-sectional view of smart tube 190 may illustrated. On one distal end of smart tube 190, a camera with light source may be shown towards the patient's trachea. At the opposite distal end of smart tube 190, light source and photosensor fibers may shows towards the patient's mouth.

It should be note that the aforementioned light source and/or light sensor may be situated at various locations in smart tube 190. In one aspect, the light source and/or light sensor may be positioned at the mouth side of smart tube 190. Here, smart tube 190 may function as light guide. Additionally, the wavelength of the light source may be tunable or adjustable so as to enhance the state space by adding additional orthogonal measurements.

In furtherance of yet another aspect of the disclosure, cuff pressure sensing may be achieved by various alternatives. One such approach would be implementing fiber sensing technologies, including, for example, Fiber Bragg Grating straining gauges. Cuff pressure sensing may also be performed at the inflatable cuff, outside the mouth. Numerous approaches may be considered here to achieve this including for example, the use of a MEMS pressure sensor(s).

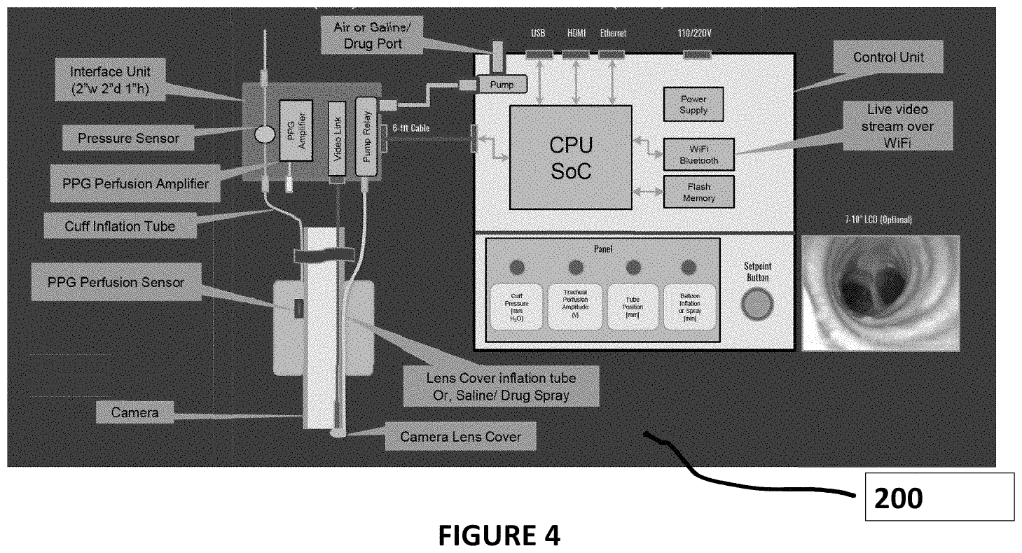

Referring to FIG. 4, a block diagram of a ventilation system 200 may illustrated. System 200 depicts various aspects of the present disclosure detailed hereinabove including features of the smart tube. It should be note that the smart tube as well as the interface unit of system 200 may be disposable for various reasons including sanitary and health sensibilities.

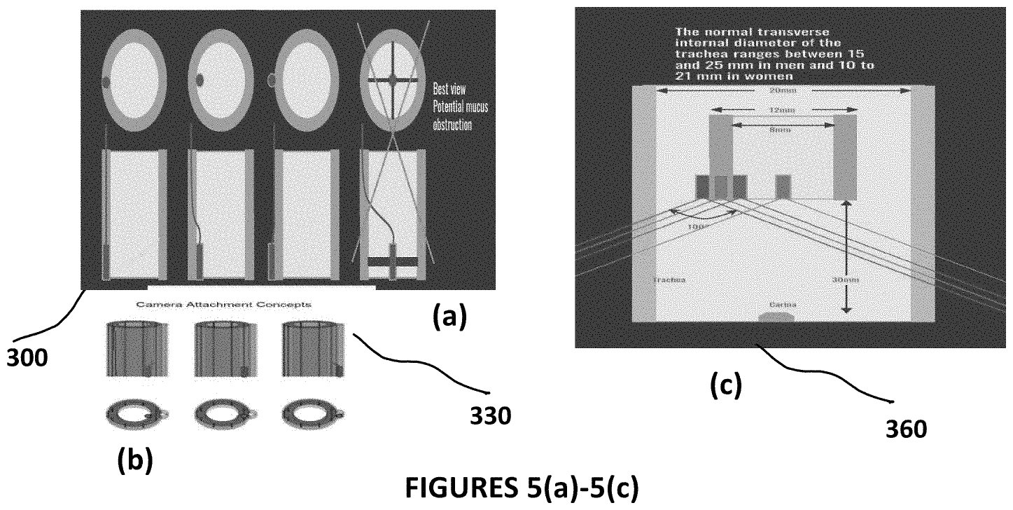

Referring to FIGS. 5(a)-5(c), a camera system 300 may illustrated. In FIG. 5(a), a series of four camera may configured in a cross sectional manner, with the right most camera, in this example, obstructed by patient mucous. As a consequence, the remaining three cameras may be empowered to provide the necessary data for full functional operation. This may be reflected in FIG. 5(b), where functional camera system 330 may illustrated. With respect to FIG. 5(c), in on aspect of the disclosure, the transverse internal diameter of the trachea may typically range somewhere between 15-25 mm in men and 10-21 mm in women.

Referring to FIG. 6, another aspect of the present disclosure may be illustrated. Here, a camera cover system 400 for the smart tube may be shown. Given that intratracheal environment may result in fogging and mucus debris that may occlude and obstruct the camera from the essential viewing area, camera cover system 400 may be essential. Camera cover system 400 may be realized by various implementations. One example may the use of a catheter like balloon positioned at the distal end of the smart tube, right above the lens. To cover the lens, the catheter like balloon may be inflated. Conversely, when light sensing may be required, the catheter like balloon may be deflated.

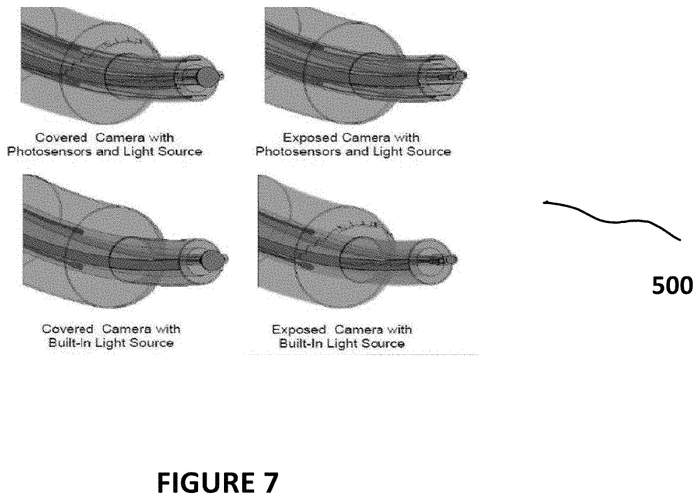

Referring to FIG. 7, another aspect of the present disclosure may be illustrated. Here, a camera and camera cover system 500 for the smart tube may be shown. In one aspect, camera and camera cover system 500 may be realized by a camera with, and, without light source having an outer diameter (“OD”) of 1 mm and 2 mm, respectively. Alternatively, a catheter like balloon that controls camera and camera cover system 500 may have a diameter of 2-3 mm when fully inflated in cover camera mode. Here, the balloon inflation tube may have an OD of about 0.5 mm. This balloon inflation tube may be extruded directly into the wall of the smart tube or, in the alternative, adjoined on the outside of the smart tube. By this arrangement, the balloon size may not disturb or interfere with the airflow of the smart tube.

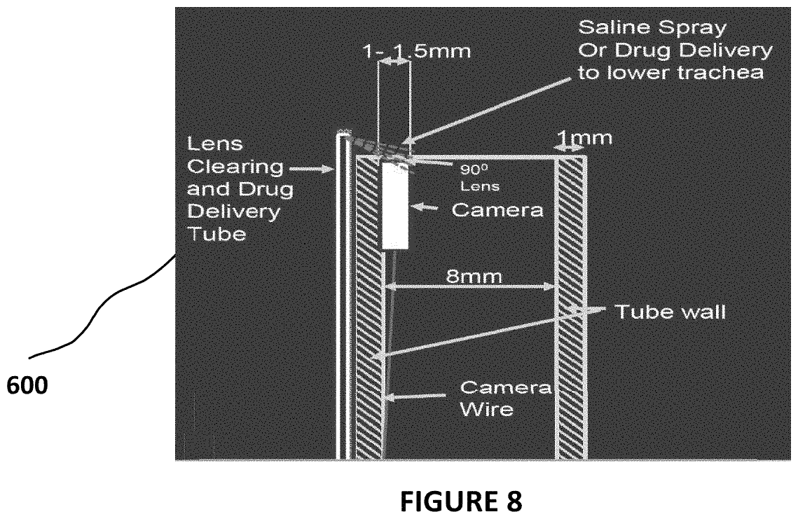

Referring to FIG. 8, another aspect of the present disclosure may be illustrated. Here, a camera lens clearance and drug delivery system 600 for the smart tube may be shown. System 600 may include that use of video and/or light sensing, in addition to a balloon inflation/deflation synchronized mechanism. A second solution may to deliver saline spray or some other solution, directed to the camera lens, to clear the lens from fogging and mucus debris arising from the patient by relying on the balloon inflation/deflation synchronized mechanism. In another aspect of the disclosure, the balloon inflation/deflation synchronized mechanism may be deployed to delivered drug therapy to the patient's lower trachea.

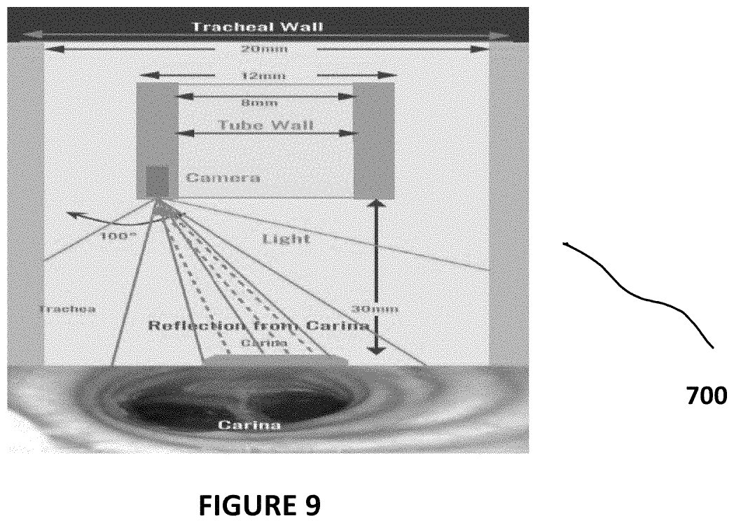

Referring to FIG. 9, another aspect of the present disclosure may be illustrated. Here, a smart tube placement and misplacement control system 700 may be shown. System 700 may use video visualization for guiding the medical professional to position the smart tube in the optimal tracheal location for the patient. System 700 may use light sensing to provide a relative distance measure between the smart tube tip and the carina. The level of light reflected from the carina may cause shading that could enable the detection of smart tube movement and, further, generate an alert to the medical professional. In another aspect of the disclosure, a miniature charge coupled device (“CCD”) or complimentary metal-oxide semiconductor (“CMOS”) type camera may be integrated with a light-emitting diode (“LED”) or an illumination source, such as optical fibers, coupled with the tip of the smart tube. Regardless of whether a CCD or CMOS type camera may be used, color visualization at high resolution may enable accurate relative distance measurements. In yet another aspect of the disclosure, miniature light sources, laser or other, of one or multiple wavelengths, and light detectors may be used for relative distance measurements.

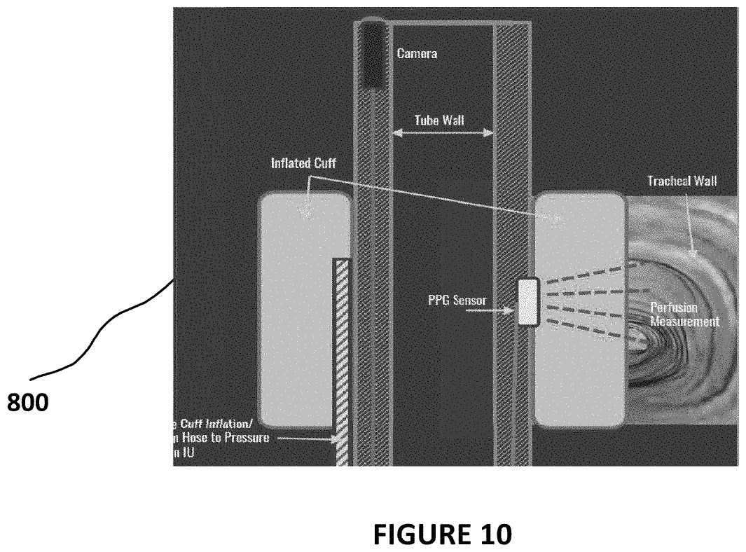

Referring to FIG. 10, another aspect of the present disclosure may be illustrated. Here, a cuff pressure control system 800 may be shown. Control system 800 controls the pressure of the inflatable cuff used in the smart tube by relating pressure to perfusion and monitoring these variables throughout intubation period.

It has been shown that intra-cuff pressures of greater than 48.9 cmH₂O may impede capillary blood flow and damage the mucosal lining of the trachea, resulting in possible long term stenosis. Conversely, intra-cuff pressures <25.5 cmH₂O may increase the risk of aspiration of mucosal secretions, thereby causing a higher incidence of ventilator associated pneumonia. As a consequence, the present disclosure contemplates coupling a sensor, such as a microelectromechanical (MEMS) pressure sensing device, to the inflatable/deflatable cuff tube. This may be centrally controlled an interface unit for the smart tube system. Further, a photoplethysmography (PPG) sensor may be connected or embedded in the smart tube at or near the cuff.

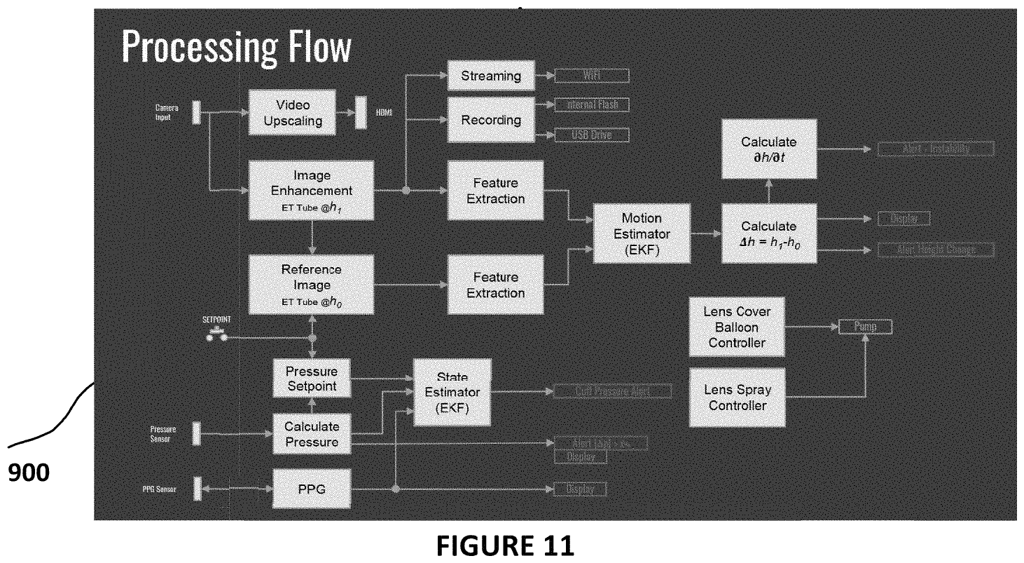

Referring to FIG. 11, another aspect of the present disclosure may be illustrated. Here, a process flow 900 may be shown. Process flow 900 depicts the sequence of steps, in one aspect of the disclosure, for a system deploying a smart tube with numerous consideration detailed and disclosed herein.

In one process is disclosed for ventilation the lungs of a patient in light of FIG. 11. The method includes the step of intubating the patient with a smart tube having a tip and a cuff. Further, the method includes providing relative positional intubation measurements of the smart tube relative to the patient. The method also includes the step of sensing pressure of the cuff. Moreover, the method includes the step of computing programmed feedback based on the relative positional intubation measurements and/or the pressure of the cuff.

In another aspect of the disclosure, the step of computing programmed feedback includes the step of generating computational real time positional guidance in the intubating of the smart tube.

In another aspect of the disclosure, the step of generating computational real time positional guidance in the intubating of the smart tube includes the step of generating misplacement signaling and/or proper placement signaling.

In another aspect of the disclosure, the method further includes the steps of measuring the computational real time positional guidance with a programmed location range, and generating a displacement alert signal if the computational real time positional guidance falls outside the programmed location range.

In another aspect of the disclosure, the step of measuring the computational real time positional guidance within the programmed location range includes the step of measuring the tip of the smart tube relative to the patient's carina.

In another aspect of the disclosure, the method further comprises the step of positioning the smart tube relative to the patient's carina within the programmed location range in response to the proper placement signaling, and/or repositioning the smart tube relative to the patient's carina within a programmed repositioning location range, in response to the displacement alert signaling.

In another aspect of the disclosure, the step of computing programmed feedback includes the step of generating computational real time cuff pressure signaling.

In another aspect of the disclosure, the method further includes the step of measuring the computational real time cuff pressure signaling with a programmed pressure range, and/or generating a real time cuff pressure signaling alert if the computational real time cuff pressure signaling falls outside programmed pressure range.

In another aspect of the disclosure, the method further includes the step of maintaining the cuff pressure within the programmed cuff pressure range in response to computational real time cuff inflation signaling.

It should be noted that other aspects of the present disclosure have been considered. Other aspects of the present disclosure may be found hereinbelow. The smart tube and system disclosed herein may provide for highly accurate and safe management of the endotracheal airway in support of mechanical invasive, lung ventilation. The smart tube and system of the present disclosure may be used to securely establish an airway whenever mechanical ventilation may be required.

In one aspect of the disclosure, the smart tube and system may:
1. Correct placement of the smart tube in the trachea above the tracheal carina;
2. Safe and highly accurate smart tube cuff inflation pressure;
3. Smart tube displacement alerts;
4. Smart tube cuff pressure change alerts; and
5. Remote monitoring of monitoring variables.

In an aspect of the present disclosure, the smart tube may include a miniature CCD or CMOS camera bundled with an LED or optical fibers illumination source. The camera may be located at the tip of the smart tube, supporting color visualization at high resolution. The video captured by the camera during the intubation process, may enable medical professionals to position the smart tube tip at optimal distance above the tracheal carina.

In an aspect of the present disclosure, the image captured by the camera may undergo an image processing step for measuring the relative distance from the smart tube tip to the tracheal carina. The rate of the measurement may be programmed by the medical professionals in a range, for example, of 1 seconds to 60 minutes.

In another aspect of the present disclosure, the camera may be programmed to alert the medical professionals monitoring the intubating patient if and when the smart tube tip may at a pre-determined safe proximity to the tracheal carina. Medical professionals monitoring the intubating patient may determine and program the optimal position of the smart tube (h₀), the position characteristics recorded by the smart tube system and used to calculate actual distance to carina (h₁) continuously at the per-programmed rate. Optimal smart tube position may be characterized by the equation: Δh=h₁−h₀=0.

In aspect of the present disclosure, the camera may be programmed to alert medical professionals of tube displacement (Δh=h₁−h₀) from the position relative to the carina 1 (h₁). The threshold for maximum displacement of the smart tube tip, (Δhₘₐₓ) may be set by the medical professionals intubating the patient, which may typically have a Δh range of ±1.5 cm.

Due to camera fogging, the smart tube may include n number (n=1 to 100) of miniature light sources at the tip of the smart tube, directed toward the tracheal carina and right and left bronchial tree take-offs, the light source may be laser or another source and of one or multiple wave lengths. The smart tube maybe connected to a photosensor, dialed for one or multiple wavelengths (wl₁₋ₓ). Changes in photo-signal intensity (pi₁₋ₓ), following the medical professional set points of h₀ and h₁, should automatically calculate tube location (wl₁₋ₓip₁₋ₓ^{set}). Displacement (Δh=h₁−h₀) from the smart tube position relative to the tracheal carina (h₁) may be calculated by changes in light intensity (Δh=wl₁₋ₓip₁₋ₓ^{set}−wl₁₋ₓpi₁₋ₓ^{move}). The threshold for maximum displacement of the smart tube tip, (Δhₘₐₓ) may already be set by the medical professional intubating the patient (typically (Δh=±1.5 cm) with the use of the camera at the time of tube placement. Light intensity relative to distance (mm) should be automatically calculated from final placement h₁.

The smart tube cuff may have a pressure sensor allowing the intubating the medical professional an accurate read of the cuff pressure (P). Cuff pressure may be controlled manually and/or automatically by a pressure cuff pump in the control unit. During smart tube placement, the cuff sensor may alert the medical professional when P>20 cm H₂O or any set alert P determined by the medical professional. During smart tube placement, the cuff sensor may alert the medical professional when P=30 cm H₂O. After inflating the cuff, the pressure at full inhalation and full exhalation (Pᵢ₀ and Pₑ₀) may be recorded by the Intubating the medical professional or by the system. The pressure sensor may alert the medical professional when change in cuff pressure (ΔP=Pᵢ₁−Pᵢ₀ [symbol] V Pₑ₁−Pₑ₀) ΔP±2 cm H₂O, or any interval set by the medical professional.

At the cuff level on the smart tube there may be a perfusion sensor, using photoplethysmography (“PPG”) sensor or speckleplethysmogram (“SPG”) to measure the volumetric variations of blood circulation (e.g., amplitude and intensity) to monitor mucosal perfusion (MP) to control cuff pressure. Alerts for over pressure (MPₘₐₓ) or under pressure (MPₘᵢₙ) may be set by the medical professional since mucosal perfusion, measured as capillary flow, differs by age, sex, tracheal diameter, tracheal mucosa thickness, inflammation and disease status. For adults, this may be measured MPₘᵢₙ at P=25.5 cmH2O for under pressure and MPₘₐₓ at P=48.9 cmH₂O for over pressure. Mucosal perfusion MPᵢ₀ and MPₑ₀ may be set automatically when Pᵢ₀ and Pₑ₀ may set.

Cuff pressure Pᵢ₀ and Pₑ₀ may be measured in relation to mucosal perfusion (MPᵢ₀ and MPₑ₀). Adjustments to cuff pressure changes (ΔP) may be in relation to ΔMP where ΔMP=MPᵢ₀ [symbol] V MPₑ₀ may >MPₘₐₓ or <MPₘᵢₙ. Alerts may be given at such changes to either manually or automatically adjust cuff pressure up or down.

The camera, light sensors, mucosal perfusion sensors and pressure sensor readouts may feed an algorithm detecting movement related to cuff pressure and cuff pressure in relation to mucosal perfusion. Changes Δh±1.0 cm & Δ P±1.5 cm H₂O & >MPₘₐₓ or <MPₘᵢₙ may result in alert, other thresholds can be programmed by the medical professional.

The system may continuously self-learn the relations between smart tube tip's position stability and changes with respiratory cycle, mucosal perfusion and cuff pressure, refining the recommended cuff pressure. Pressure sensor data, mucosal perfusion sensor data, camera's live video and light sensors may be displayed on an integrated display and/or on an external HDMI video monitor. In addition, the entire video and cuff pressure statistics can be recorded on an external USB drive or streamed live over wired Ethernet or Wi-Fi to a remote unit.

The smart tube (tube, camera, light source and sensor, MP sensor and P sensor) may disposable. The IU may or may not be disposable. The CU may not disposable. By example, various arrangements to make a working smart tube system model can include the follow:
1. Disposable smart tube made of clear polyvinyl chloride (PVC) and have a high-volume low-pressure cuffed design that conforms to the shape of the trachea. The smart tube may made with transparent plastic that allows the visualization of exhalational condensation (“breath fogging”), secretions, and other foreign materials within the smart tube. The cuff near the distal end of the smart tube may inflated usually with air to create an airtight seal. The smart tube may be made of other materials, including nylon, silicone, and Teflon. Since plastic may not radio opaque, the plastic tubes may have a line of radio opaque material that makes them more visible on a chest X Ray.
   Tubes may be manufactured in multiple sizes. The most common size may be 6 mm internal diameter for women and 8 mm for men. For children the most common internal diameter may be 4 mm. The smart tube wall size may be most commonly 1 mm, making the outside diameter 8 mm, 10 mm, 6 mm in the most common designs for women, men and children. The proximal end, connecting to the ventilator has a universal connecter, with an approximate 15 mm diameter.
   The smart tube cuffs may made of clear nylon. Some cuffs may made of silicone. They may be designed to either have high pressure with small surface contact (O-ring design), or most commonly, low pressure large surface contact (tube-seal design).
   The cuff may be connected to a 1 mm flexible tube attached externally to the smart tube. At the end of the smart tube there may a port for inflation and a mechanical release valve to allow for cuff deflation. Cuff inflation may be done by attaching a syringe to the cuff tube proximal end. Cuff inflation may be done by “feel”.
   The CCD or CMOS camera integrated with LED or optical fibers illumination source to the tip of the smart tube may be attached to, or embedded in, the PVC ET tube and connected with the proper wiring and fiberoptic cable, with a diameter of 1.0 mm-1.6 mm. The electrical wires and optical fibers may be plugged or connected to the interface unit. Integrating the camera with the smart tube can be accomplished in a few ways
      1) Attached to the exterior of the smart tube;
      2) interior of the smart tube;
      3) center of tube (using cross links); or
      4) embedded in the wall of the smart tube.
   Ultra-thin wires from the camera can be 1) embedded inside the smart tube wall or 2) attached to the exterior of the smart tube. In case of fiber optics illumination, 1 to 8 thin plastic optical fibers (POFs) may be extended from the camera tip using the same routing as the electrical wires.
   In addition to the camera, one or more optical fiber light sources illuminating the smart tube front field-of-view and one or more optical fiber surfaces collecting the reflected light, may be embedded in the smart tube. By means of reflected light intensity analysis, displacement of the smart tube in and out of a pre-set distance from carina 1, can be estimated and trigger an alert if needed as set by the intubating the medical professional.
   As an alternative to the reflective fiber pairs, the camera can be used as an array of large area photodetector by calculating the average intensity in any predefined zone of the camera by averaging the pixels readout. This method of measuring reflected light intensity from different zones in the field-of-view, may relatively insensitive to lens fogging, condensation or particulate contamination.
   The cuff inflation/deflation tube may be connected to an external pressure sensor in the interface unit or a fiber Bragg grating (FBG) sensor embedded in the smart tube at the cuff level, or a microelectromechanical (MEMS) pressure sensor.
   A photoplethysmography (PPG) sensor or speckleplethysmogram (SPG) may be connected or embedded in the smart tube at the cuff level.
2. Interface Unit (IU): The interface unit may or may not be disposable. The IU may be located in close proximity to the patient and the proximal/exterior end of the smart tube. The IU may be used to process the pressure sensor signal, the PPG or SPG signal, photosensors signal and the raw camera video stream. It includes an interface connector for connecting the smart tube to the Control Unit using a dedicated cable. It also includes an air outlet to connect the cuff inflation hose and air inlet to connect the inflating pressure source (e.g. a syringe) and the manual cuff pressure release valve.
3. Control Unit (CU): The CU may be located next to the ventilator's monitor. The CU may be connected to the IU using a single cable, to a power source and Optionally, to the LAN. The unit consists of central processing unit that executes the video processing algorithms, enable recording and streaming of the data and run machine learning algorithms. In addition to that, the CU may include an integrated-display unit, visual and sound alerts indicators. For an extended duration of intubation (days), The CU can be connected to a local area network (LAN) using Ethernet or Wi-Fi where the real time data can be streamed for remote monitoring by the medical staff, typically monitoring multiple intubations.

The smart tube cuff may have a pressure sensor allowing the intubating the medical professional an accurate read of the cuff pressure (P). Cuff pressure may be controlled manually and/or automatically by smart tube operating sequence:
1. Plug the CU to electrical source;
2. Plug smart tube to CU—if IU unit may not disposable, plug camera plug and attach smart tube cuff inflation tube;
3. Set camera's optimal position of the smart tube (h₀);
4. Set lower and upper cuff pressure limit alerts (Pₐ₁ and Pₐ₂);
5. Set pressure P for overpressure (MPₘₐₓ) or under pressure (MPₘᵢₙ);
6. Make sure all other tools may ready for intubation such as the laryngoscope;
7. Ensure video camera may working and can be viewed on the monitor;
8. Ensure light source and photosensors may working and can be viewed on the monitor;
9. Ensure cuff pressure may working and can be viewed on the monitor;
10. Ensure mucosal perfusion sensor may working and can be viewed on the monitor;
11. Start intubation, following institutional intubation protocol;
12. Set final smart tube position to calculate actual distance to carina (h₁);
13. Inflate smart tube cuff; and
14. Set smart tube cuff pressure at full inhalation and full exhalation (Pᵢ₀ and Pₑ₀).

In one aspect of the present disclosure, the following details have been considered in assembling a prototype. For the smart tube, and for each part considered by brand, type, specifications, certifications, including ISO, FDA, for example:
1. Clear PVC, Teflon, Silicon or Nylon tube with a line of radio opaque material;
2. Inside smart tube diameters range from 4 mm-11 mm;
3. Smart tube wall may range from 1 mm-1.5 mm;
4. Outside smart tube diameter may be 6 mm-14 mm;
5. Proximal (outside) end may be attached to a universal intubation line connector 15 mm diameter;
6. Inflatable clear cuff made of nylon or silicone;
7. Cuff may be connected to 1 mm-1.5 mm PVC, Teflon, silicone or nylon tube;
8. CCD or CMOS camera may either embedded or attached to the smart tube wall;
9. CCD or CMOS camera wires and fiberoptic line may either embedded or attached to the smart tube wall;
10. One or multiple light sources, laser or other at one or multiple wavelengths embedded in smart tube;
11. One or multiple photosensors embedded in smart tube;
12. Optional: FBG or MEMS sensor may be embedded in smart tube wall at the level of the cuff;
13. Optional: An additional FBG sensor may embedded in the smart tube wall at the level above the cuff to compensate FBG response to temperature gradients;
14. Optional: PPG or SPG sensor may embedded in the smart tube wall at the level of the cuff; and
15. Optional: A silicone or Teflon O-ring umbrella covering the glottis.

For the interface unit, and for each part considered by brand, type, specifications, certifications, including ISO, FDA, for example:
1. Pressure sensor;
2. CCD or CMOS raw data connection;
3. Light sources and photosensors data connection;
4. smart tube cuff inflation tube connected to the pressure sensor;
5. smart tube cuff manual deflation valve connected to the inflation tube;
6. smart tube cuff inflation tube port for manual syringe inflation;
7. IU to CU dedicated cable;
8. Optional: FBG or MEMS sensor connector; and
9. Optional: PPG or SPG sensor connector.

For the control unit, and for each part considered by brand, type, specifications, certifications, including ISO, FDA, for example:
1. Central processing unit for video;
2. Light processing unit;
3. Pressure processing unit;
4. Mucosal perfusion processing unit;
5. Integrated processing unit;
6. Integrated visual display unit;
7. Light alerts;
8. Sound alerts;
9. Ethernet port;
10. USB port;
11. Wi-Fi radio;
12. Optional: FBG, MEMS and PPG or SPG processing unit;
13. Optional: Air pump (0 cm H₂O-50 cm H₂O), pressure gauge (0 cm H₂O-50 cm H₂O); and
14. Optional: Smart tube cuff inflation tube connection to pump.

For the cables, CU to wall electrical wire was considered. With respect to HDMI display, and for each part considered by brand, type, specifications, certifications, including ISO, FDA, for example:
1. Wiring Diagram;
2. Wiring Logic;
3. Control Parameters;
   a. Distance;
   b. Pressure;
   c. Perfusion;
4. Display Logic;
   a. Start/Stop switch. Controls the start and stop of smart tube;
   b. Video;
   c. Distance;
   d. Pressure; and
   e. Perfusion.

In another aspect of the disclosure, the camera, light sensors, mucosal perfusion sensors and pressure sensor readouts may feed an algorithm detecting movement related to cuff pressure and cuff pressure in relation to mucosal perfusion. Changes Δh±1.0 cm & Δ P±1.5 cm H₂O & >MPₘₐₓ or <MPₘᵢₙ may result in alert, other thresholds can be programmed.

The miniature CCD or CMOS camera may easily fog by humidity of lung gases. The camera may get obstructed by mucus or tracheal epithelial mucosal folds. More so, the video quality may not be sufficient for accurate distance measurements. Fogged or obstructed video view may result in deteriorated signal during prolonged ventilation.

In another aspect of the disclosure, the following have been taken into consideration:
1. Laser source with a reflection detector instead or in addition to the CCD or CMOS camera; and
2. The camera readout while lens may clean or fogged/contaminated, can be used to measure the intensity of reflected light from the carina in order to estimate the smart tube tube's tip movement relative to a setpoint;
3. Smart tube cuff pressure may be monitored by the medical professional by “feel.” There may be no control system providing actual cuff pressure measurement. More so, cuff pressure may both individual and dynamic based on the individual patient's trachea size, airway mucosal inflammatory state, inhalation and exhalation changes of tracheal diameter and breathing fluctuations. In addition, providing a proper seal may weighed against balancing two competing clinical requirements: 1) high pressure may impede capillary blood flow resulting in trachea mucosal lining damage (long term stenosis) and, 2) low pressure may increase the risk of aspiration of mucosal secretions (causing a higher incidence of ventilator associated pneumonia) or result in inadequate ventilation from loss of pressure. In order to increase smart tube cuff pressure control and automation:
   a. We may include a Fiber Braggs Gratings (FBG) or MEMS pressure sensor, embedded in the smart tube wall at the cuff level, to monitor cuff pressure;
4. Smart tube cuff pressure release—we may use photoplethysmography (PPG) or SPG sensors (a light source and a photodetector at the surface of skin) to measure the volumetric variations of blood circulation in order to monitor mucosal perfusion to control cuff pressure;
5. We may automate cuff pressure based on pressure mucosal perfusion algorithm;
   a. We may include a pump, pressure sensor and release valve to the smart tube cuff inflation/deflation line to automatically control smart tube cuff pressure; and
6. We may include a silicone or Teflon O-ring umbrella covering the glottis.

It should be understood that the figures in the attachments, which highlight the structure, methodology, functionality and advantages of this disclosure, may presented for example purposes only. This disclosure may sufficiently flexible and configurable, such that it may be implemented in ways other than that shown in the accompanying figures.

What is claimed is:

1. A system for ventilation the lungs of a patient comprising:
a smart tube for intubation comprising a tip and a cuff;
at least one camera, coupled with the smart tube, for at least one of:
providing relative positional intubation measurements of the patient; and
providing intubation images of the patient;
at least one sensor, coupled with the smart tube, for at least one of:
sensing the inflation of the cuff; and
sensing pressure of the cuff;
a computing system, coupled with the at least one camera, the at least one sensor and the smart tube, for computing programmed feedback based on at least one of:
the relative positional intubation measurements;
the intubation images;
the inflation of the cuff; and
the pressure of the cuff.

2. The system of claim 1, wherein the programmed feedback generates computational real time positional guidance in the intubation of the smart tube.

3. The system of claim 2, wherein the computational real time positional guidance generates at least one of misplacement signaling and proper placement signaling.

4. The system of claim 3, wherein the computing system generates a displacement alert signal if the computing system measures the computational real time positional guidance outside a programmed location range.

5. The system of claim 4, wherein the computing system generates the proper placement signaling if the computing system measures the computational real time positional guidance within a programmed location range of the tip of the smart tube relative to the patient's carina.

6. The system of claim 5, wherein the computing system further comprises a programmed servo system for at least one of:
positioning the smart tube relative to the patient's carina within the programmed location range in response to the proper placement signaling; and
repositioning the smart tube relative to the patient's carina within a programmed repositioning location range, in response to the displacement alert signaling.

7. The system of claim 1, wherein the programmed feedback generates computational real time cuff pressure signaling.

8. The system of claim 7, wherein the computing system generates a real time cuff pressure signaling alert if the computing system measures the computational real time cuff pressure signaling outside a programmed cuff pressure range.

9. The system of claim 8, wherein the computing system further comprises a programmed servo system for maintaining the cuff pressure within the programmed cuff pressure range in response to computational real time cuff inflation signaling.

10. The system of claim 1, wherein the smart tube, the at least one camera, the at least one sensor, and the computing system are integrated within a singular housing.

11. A method for ventilation the lungs of a patient comprising:
intubating the patient with a smart tube having a tip and a cuff;
providing relative positional intubation measurements of the smart tube relative to the patient; and
sensing pressure of the cuff;
computing programmed feedback based on at least one of:
the relative positional intubation measurements; and
the pressure of the cuff.

12. The method of claim 11, wherein the step of computing programmed feedback comprises the step of generating computational real time positional guidance in the intubating of the smart tube.

13. The method of claim 12, wherein the step of generating computational real time positional guidance in the intubating of the smart tube comprises generating at least one of misplacement signaling and proper placement signaling.

14. The method of claim 13, further comprising the steps of:
measuring the computational real time positional guidance with a programmed location range; and
generating a displacement alert signal if the computational real time positional guidance falls outside the programmed location range.

15. The system of claim 14, wherein the step of measuring the computational real time positional guidance within the programmed location range comprises the step of measuring the tip of the smart tube relative to the patient's carina.

16. The method of claim 15, further comprising at least one of:
positioning the smart tube relative to the patient's carina within the programmed location range in response to the proper placement signaling; and
repositioning the smart tube relative to the patient's carina within a programmed repositioning location range, in response to the displacement alert signaling.

17. The method of claim 11, wherein the step of computing programmed feedback comprises the step of generating computational real time cuff pressure signaling.

18. The method of claim 17, further comprising the steps of:
measuring the computational real time cuff pressure signaling with a programmed pressure range; and
generating a real time cuff pressure signaling alert if the computational real time cuff pressure signaling falls outside programmed pressure range.

19. The method of claim 19, further comprising the step of:
maintaining the cuff pressure within the programmed cuff pressure range in response to computational real time cuff inflation signaling.
